(19) United States
(12) Patent Application Publication
(10) Pub. No.: US 2011/0059456 A1
(43) Pub. Date: Mar. 10, 2011

(54) System for the Cell-Specific and Development-Specific Selection of Differentiating Embryonic Stem Cells, Adult Stem Cells and Embryonic Germline Cells

(75) Inventors: Bernd FLEISCHMANN, Koln (DE); Heribert Bohlen, Koln (DE); Jürgen Hescheler, Koln (DE); Eugen Kolossov, Koln (DE)

(73) Assignee: Axiogenesis AG, Cologne (DE)

(21) Appl. No.: 12/876,772

(22) Filed: Sep. 7, 2010

Related U.S. Application Data
(62) Division of application No. 10/451,816, filed on Dec. 15, 2003.

(30) Foreign Application Priority Data
Dec. 27, 2000 (DE) 100 65 352.9
Jul. 27, 2001 (DE) 101 36 702.3

(51) Int. Cl. C12Q 1/68

(52) U.S. Cl. 435/6

(57) ABSTRACT

The invention relates to a system for selecting differentiating embryonic or adult stem cells or embryonic germline cells in a cell-specific and development-specific manner, using a combination of resistance genes and detectable reporter genes under the common control of a cell-specific and/or development-specific promoter.

The invention relates to recombinant embryonic stem cells, embryonic germline cells and adult stem cells which contain a gene for a non-cell-damaging, detectable protein as well as a resistance gene, methods for the preparation of the cells as well as further embodiments.

The in vitro cardiomyogenesis of differentiating embryonic stem cells (ES) in culture was suggested as an unlimited source of heart muscle cells for transplantation in the replacement therapy of irreversibly damaged heart tissue (Klug et. al., 1996). One of the major obstacles for the practical implementation of this approach is the relatively low yield of differentiated, ES-derived heart muscle cells, which usually constitute no more than 1-3% of a differentiating overall ES-cell population (Muller M. et al., 2000).

Furthermore the still existing non differentiated ES-donor cells pose a potential threat for the recipient in later stages of differentiation regarding the development of tumours. Therefore the aim of developing an effective and highly specific selection method is considered to be a milestone in the cell therapy of heart disorders. It had already been demonstrated earlier that a population enriched in heart muscle cells can be selected successfully from genetically modified ES-cells which are stably transfected with a transgene of a drug resistance gene of aminoglycosid-phosphate-transferase (α-MHC-Neo) controlled by a α-heart-myosine-heavy-chain-promoter (Klug et al., 1996). This work further showed the potential problems for the development of this approach into an effective procedure in large scale:

a) The treatment with a selective drug (G418) was carried out in an adherent culture of differentiating ES-cells, whereas from the point of view of effectivity as well as of technological feasibility, the optimal approach would be the application of the selective drug directly onto a suspension of aggregates of ES-cells—embryoid bodies (embryoid bodies=EBs) (Wobus et al., 1991).

b) The further experiments regarding the introduction of genetically selected cells into the heart of recipient animals is made more complicated significantly by the work to demonstrate the fate of the introductions in absence of specific viability markers for donor cells.

DE-A-19727962 describes embryonic stem cells of non-human mammals, which are stably transfected with a DNA construct which comprises a DNA sequence that encodes a non-cell-damaging fluorescent protein, wherein said DNA sequence is under control of a cell- and/or development-dependent promoter (Kolossov et al., 1998). Such recombinant ES-cells exhibit the following disadvantages:

1. Though specific cell types can be provided in vitro with this method, nevertheless the purification of these vitally stained cells is difficult. On the one hand, this can be explained by the fact that the cells of interest (e.g. cardiomyozytes) account only for about 1-3% of the cells generated in EBs. On the other hand, cell purification methods (e.g. fluorescence activated cell sorting, FACS) are ideally suited for immunological cells. On purification, however, of e.g. cardiomyocytes many cells perish or are irreversibly damaged.

2. Further it turned out that with the hygromycin purification method on plated EBs the non-hygromycin resistant cells are difficult to remove even after 7-14 days of selection. Despite the use of hygromycin as a selection marker beforehand a generation of-tumours occurred. This applies similarly to a selection with neomycin.

It is an object of the present invention to provide a novel system for both the selection and the extraction of cells, respectively, from a differentiating culture of embryonic stem cells, embryonic germline cells and adult stem cells which avoid the above mentioned problems. As “system” a combination of selection methods, cells and use of the cells and methods particularly in the medical field is to be understood, as described in the present application. This object is achieved by embryonic stem cells, embryonic germline cells and adult stem cells of claim 1. Preferred embodiments of the invention are described in the claims following claim 1.

The invention discloses a system for the cell- and/or development-specific selection of differentiating embryonic stem cells, embryonic germline cells and adult stem cells by the combined application of (drug) resistance and detectable reporter genes under the common control of a cell- and/or development-specific promoter.

The present invention is first illustrated in general and subsequently by means of examples based on the genetic selection of heart cells from a differentiating culture of embryonic stem cells that are transfected with two kinds of vectors. It is emphasized that the invention is not limited to these particular embodiments, but is applicable to all 3 germlayer derived cell types, i.e. endoderm, mesoderm and ectoderm and cells derived therefrom due to the pluripotency of the stem cells and germline cells, respectively. A person skilled in the art is able to vary the invention within the scope of the appended claims having regard to the following description and his general knowledge.

According to the invention the information for at least one resistance gene and for at least one detectable reporter gene encoding e.g. a non-cell-damaging detectable protein, is introduced into embryonic stem cells, embryonic germline cells and adult stem cells. The information for both genes can be available on one or distributed onto two vectors. Crucial is that the expression of the gene for the detectable, e.g. fluorescent protein as well as for the resistance gene is under. control of one and the same promoter.

According to the invention, the promoters are selected from cell-specific promoters and development-specific promoters. Cell- and tissue-specific promoters, respectively, refer to those that are active in specific cell populations and tissues, respectively. Thereto belong e.g. neuronal cells, endothelial cells, skeletal muscle cells, cells of the smooth muscle tissue as well as keratinocytes. Particularly preferred are heart muscle cells (cardiomyocytes).

Further examples for tissue specific promoters are those, which are active in glia cells, hematopoietic cells, neuronal cells, preferably embryonic neuronal cells endothelial cells, cartilage cells or epidermal cells as well as insulin secreting β-cells. “Tissue-specific” is to be subsumed under the term “cell-specific”.

Examples for heart specific promoters are: Nkx-2.5 (specific for very early cardiomyocytes and mesodermal precursor cells, respectively, (Lints et al., 1993); human-cardiac-α-actin (specific for heart tissue, (Sartorelli et al., 1990), MLC-2V (specific for ventricular heart muscle cells (O'Brien et al., 1993) and WO-A-96/16163).

Further examples for non-heart specific promoters are: PECAM1, FLK-1 (endothelium), nestine (neuronal precursor cells), tyrosin-hydroxylase-1-promoter (dopaminergic neurons), smooth muscle α-actin, smooth muscle myosin (smooth muscles), α1-fetoprotein (endoderm), smooth muscle heavy chain (SMHC minimal promoter (specific for smooth muscles, (Kallmeier et al., 1995).

The term development-specific promoter refers to promoters that are active during certain points of time during development. Examples for such promoters are the β-MHC promoter that is expressed during embryonic development in the ventriculum of the mouse and is superseded by the α-MHC promoter in the prenatal phase. NKx2.5, a promoter during the early mesoderm/heart development, atrial-natriuretic-factor, a marker of the early embryonic heart with exception of the pacemaker which is down regulated also in later developmental stages, Flk-1, an endothelium-specific promoter that is active during the early vasculogenesis, intron 2-segment of the nestine gene that is expressed in neuronal precursor cells (embryonic neurons and glia cells) and adult glia cells (partially still able to divide) (Lothian and Lendahl, 1997).

According to the invention promoter relates to a DNA sequence region that controls the transcription of a gene. It comprises in one embodiment at least a minimal sequence that is located upstream of the start codon and comprises the binding site for the RNA polymerase for the initiation of transcription. This minimal sequence can be supplemented by further functional DNA sections, particularly enhancer. Also applicable are regulatory elements that are located in the intron regions and might be located downstream of the gene to be transcribed. In that case, the transcription rate can be controlled e.g. by other enhancer elements, that per se do not have an activity. Also promoter constructs can be used, wherein a per se non-constitutive active element (heat shock protein enhancer) is utilised with an enhancer segment of the gene, which is derived from the intron.

In a further embodiment of the invention, development-specific promoters are used that allow a selection for e.g. mesodermal cells. Applicable promoter elements which control the transcription of the resistance gene and of the gene for the detectable protein, are NKx2.5, ANF and brachyuria promoters. After detection of cells expressing the detectable protein, which can be mesodermal cells if e.g. a mesodermal-specific promoter was used, the selection agent appropriate for the resistance gene is added and mesodermal precursor cells are selected for. By transcription of the genes for the detectable protein and of the resistance gene controlled by a common promoter element, non-differentiated cells, e.g. embryonic pluripotent stem cells can be eliminated in a highly specific manner and thereby the possibility of a later development of tumours is considerably reduced. The mesodermal cells so obtained can be implanted into the respective tissue and differentiate further there, e.g. after implantation in a predamaged heart area into heart cells. On the one hand, this approach allows the production of large amounts of prepurified precursor cells and on the other hand a further differentiation after implantation under native conditions.

In a similar manner it is possible to select for endodermal or ectodermal cells by means of endodermal- or ectodermal-specific promoters.

Examples for mesodermal cells are all muscle cell types (heart muscle, skeletal muscle and smooth muscle cells), hematopoetic cells and endothelial cells. Examples for ectodermal cells are skin cells, neurons and glia cells; examples for endodermal cells are epithelial cells of the gastrointestinal tract.

By means of the specific promoters for the above-mentioned cell types and use of the method according to the invention and the cells according to the invention, a highly specific development occurs into these endodermal, ectodermal and mesodermal cells and tissues, respectively, wherein the expression of the reporter gene and the resistance gene controlled by one and the same promoter ensures a maximal level of safety, because, on the one hand, the non-differentiated pluripotent embryonic stem cells and, on the other hand, also other tissue types are eliminated.

According to the invention the reporter gene encodes e.g. a non-cell-damaging detectable protein, in one embodiment a fluorescent protein. Such non-cell-damaging fluorescent proteins are known per se.

According to the present invention, the green fluorescent protein (GFP) from the jellyfish Aequorea victoria (described in WO-A-95/07463, WO-A-96/27675 and WO-A-95121 191) and its derivates “Blue GFP” (Heim et al., Curr. Biol. 6 (2): 178-182 (1996) and Redshift GFP” (Muldoon et al., Biotechniques 22 (1): 162-167 (1997)) can be used. Particularly preferred is the Enhanced Green Fluorescent Protein (EGFP). Further embodiments are the yellow and the cyan fluorescent protein (YFP, CFP). Further fluorescent proteins are known to the person skilled in the art and can be used according to the invention as long as they do not damage the cells. The detection of fluorescent proteins takes places through per se known fluorescence detection methods.

Alternatively to the fluorescent proteins, particularly in in vivo applications, other detectable proteins, particularly epitopes of those proteins, can also be used. Also the epitope of proteins, though able to damage the cell per se, but whose epitopes do not damage the cells, can be used.

Preferably, it concerns epitopes localized on the cell surface, which allow a simple detection, e.g. by fluorescence labelling and imaging methods (magnetic particles), respectively, in combination with antibodies. Those proteins and their epitopes, respectively, are selected for in vivo applications preferably such that they are immunologically compatible to the host, that means that they do not induce rejection. Also preferably applied are transgenic epitopes of proteins that are not linked to intracellular signal cascades, particularly surface epitopes of CD8 or CD4. A further example are epitopes of receptors. It is important that it concerns those proteins and their epitopes, respectively, which are noch present, i.e. not expressed in the cell, e.g. the heart cell, that was obtained by differentiation and selection from the stem cells and germline cells, respectively, transfected with a vector according to the invention. Any proteins can be used, which are not expressed in the differentiated and selected cell, e.g. the heart cell or transgenic epitopes that are specifically detectable, and thus are not expressed in the selected cell. These proteins and epitopes are called cell marker, cell marker gene or reporter genes, respectively. The detection of theses detectable proteins and epitopes, respectively, can e.g. result from antibodies that bind specifically to these detectable proteins and epitopes, respectively, and that can be identified by e.g. fluorescence mediated methods or imaging procedures. An example are anti-CD8 or anti-CD4-fluorescence-conjugated cell surface antibodies and ferromagnetic-particle-conjugated antibody components, respectively. As an additional technique for the purification, which allows the highest degrees of purity, the cell sorting is applicable. Having already highly enriched the desired differentiated cells after addition of the selection agents, e.g. of the antibiotic puromycin, the cells can be purified further by means of MACS sorting up to 99%.

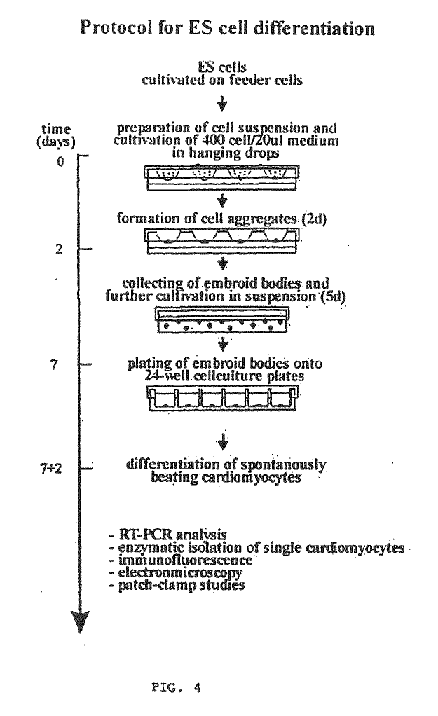

The embryonic or adult stem cells and the embryonic germline cells are in a preferred embodiment of the invention available in form of aggregates that are known as embryoid bodies. FIG. 4 shows a protocol to obtain embryoid bodies. The preparation takes place preferably with the “hanging drop” method or by methylcellulose culture (Wobus et al., Differentiation (1991) 48, 172-182).

Alternatively, spinner flasks (stirring cultures) can be used as culture method. Therefore, the undifferentiated ES-cells are introduced into stirring cultures and are mixed permanently according to an established procedure. Therefore, 10 million ES-cells are introduced into 150 ml medium with 20% FCS and are stirred constantly with a rate of 20 rpm., wherein the direction of the stirring motion is changed regularly. 24 hours after introduction of the ES-cells an extra 100 ml medium with serum is added and thereupon 100-150 ml of the medium is exchanged every day (Wartenberg et al., 2001). Under these culture conditions large amounts of ES-cell-derived cells, i.a. cardiomyocytes, endothelial cells, neurons etc. depending on the composition of the medium can be obtained. The cells are selected by means of the resistance gene either still within the stirring culture or after plating.

Alternatively, the EBs differentiated in the hanging drop might be not plated, but kept simply in suspension. Even under these conditions a progression of the differentiation could be observed experimentally. However, it was surprisingly shown that the application of the resistance gene led to a much faster dying of the non-cardiomyocytes and that the remaining cardiomyocytes subsequently began to beat spontaneously. This experimental finding clearly indicates that cardiomyocytes do not need specific signals from the surrounding tissue for their survival and that further the puromycin-selectioned cardiomyocytes are functionally intact. The washing off of the non-cardiomyocytes is also clearly facilitated, since with mechanical mixing alone and addition of low concentration of enzyme (e.g. collagenase, trypsin) a single cell suspension is achieved with easy washing off of the non-cardiomyocytes.

The embryonic stem cells are derived from mammals, particularly preferred from rodents, e.g. mice, rats or rabbits. Particularly preferred ES-cells are D3 cells (Doetschmann et al., 1985) (Doetschmann et al., J. Embryol. Exp. Morphol. 87, 27 (1985)), R1 cells (Nagy et al., PNAS (1995)), E14 cells (Handyside et al., Roux Arch. Develop. Biol. 198, 48 (1989)), CCE cells (Bradley et al., Nature 309, 255 (1985)) (of course other ES-cells can be used that are already known or are to be developed in future) and P19 cells (these are teratocarcinoma-derived cells with limited characteristics (Mummery et al., Dev. Biol. 109, 402 (1985)).

In a further preferred embodiment, embryonic stem cells of primates are used, as described e.g. by Thomson, J. A. et al., 1995.

In a particularly preferred embodiment, human embryonic stem cells are used. The preparation of these embryonic stem cells is already established (Thomson J A et al., 1998). Therefore, the inner cell mass of a blastocyst is obtained and plated onto mouse feeder cells. After successful propagation the cells are split and their stem cell properties are analysed by means of RT-PCR for specific stem cell genes, by immunohistochemistry for identification of specific proteins and by metabolic products. Furthermore, the stem cell status can be determined by in vitro differentiation into different cell types and propagation and splitting over several passages.

Alternatively to embryonic stem cells also embryonic germline cells (EG) (Shambott M J et al., 1998) are suitable, which are obtained from an early embryo and can be cultivated and differentiated like embryonic stem cells in the time following.

The invention is also applicable to adult stem cells. It is referred to the literature of Anderson et al., 2001, Gage, F. H., 200 and Prockop, D. J., 1997, wherein the extraction and culture of those cells is described.

Resistance genes per se are known. Examples for these are nucleoside- and aminoglycoside-antibiotic-resistance genes, e.g. puromycin (puromycin-N-acetyltransferase), streptomycin, neomycin, gentamycin or hygromycin. Further examples for resistance genes are dehydrofolate-reductase, which confers a resistance against aminopterine and methotrexate, as well as multi drug resistance genes, which confer a resistance against a number of antibiotics, e.g.against vinblastin, doxorubicin and actinomycin D. Particularly preferred is a construct, which confers a puromycin resistance. The terms resistance gene and drug or active substance resistance gene are used synonymously herein and refer to e.g. a gene encoding an antibiotic resistance in each case. Other genes encoding drug and active substance resistances, respectively, can be used as well, e.g. the DHFR-gene.

Instead of the resistance genes other selectionable marker genes can be used, which allow a specific selection of the cells containing a construct of the invention and that can be applied in vivo, without impairing the survival of the patients. Suitable genes are available to the person skilled in the art.

In the first example, the genes for the detectable protein and the resistance gene are located on two different constructs. The use of two different vectors, wherein the resistant gene is located on the first vector and the reporter gene on the second vector, e.g. EGFP, wherein both are controlled by a cell- and tissue-specific, respectively, or development-specific promoter, e.g. by the α-MHC-promoter, demonstrates the manifold advantages described in the present application, which are suitable for certain purposes. However, further experiments showed that this system is surprisingly also associated with certain disadvantages, namely with the formation of cells though resistant against the resistance gene, but containing sub-cell-clones within, that do not express the reporter gene, e.g. EGFP, thus are e.g. EGFP negative. Such sub-clones might be a potential source for teratocarcinomas, since not all non-specific cells, also e.g. non-cardiomyocytes, are eliminated even on application of the antibiotic. This might potentially lead to the survival of fast proliferating ES-cells which can form tumours.

In the experiments carried out, it was observed that indeed EGFP negative cells can survive even after puromycin exposition for up to 15 days. A possible reason for this observation is that the two vectors used were introduced into the cell in a double-transfection. Then, these vectors integrate at random into the host genome, partially at different sites of the native genome and therefore get under the influence of different genes and their control sequences, which possess different transcription activities.

Therefore, in a further embodiment of the invention (Example 2) the reporter gene and the resistance gene were arranged on one vector construct under control of one promoter. In the present Example 2 the puromycin-resistance-cassette (Pac) as well as the reporter gene EGFP were both brought under common control of the tissue specific promoter α-MHC. The major advantage of this system is the very low incidence of resistant cells, that are not cell- or tissue- or development-specific. For example, the probability for the occurrence of puromycin-resistant cells which are not heart cells is very low. This appears to be due to the fact that the Pac-cassette and the EGFP-gene integrate only at one or a few sites into the host genome and are therefore not subject to the influence of different activity rates of the respective up- or downstream located gene structure. By further selection of the obtained clones, it is possible to obtain a virtually pure cell system. This evaluation occurs using the EGFP-expression. In this regard, it is pointed our again that EGFP, α-MHC and Pac are a matter of exemplary embodiments of the invention. A person skilled in the art might make modifications having regard to the alternatives described in the above application.

The introduction of the vector construct or constructs into the embryonic stem cells occurs in a known manner, e.g. by transfection, electroporation, lipofection or with the help of viral vectors.

For the selection for stably transfected ES-cells vector constructs contain a further selectable marker gene, which confers e.g. a resistance against an antibiotic, e.g. neomycin. Of course, other known resistance genes can be used as well, e.g. the resistance genes described above in association with the fluorescent protein encoding genes. The selection gene for the selection for stably transfected ES-cells is under the control of a different promoter than that which regulates the control of the expression of the detectable protein. Often constitutively active promoters are used, e.g. the PGK-promoter.

The use of a second selection gene is important, for the ability to identify the successfully transfected clones (efficiency is relatively low) at all. Otherwise a smothering majority of non-transfected ES-cell would exist and during differentiation e.g. no EGFP positive cells could be detected.

After transfection the constructs are stably integrated into the native DNA. After activation of intracellular signals that are either cell-specific and/or development-specific, the promoter is activated and the detectable protein as well as the (first) resistance gene is expressed. It is not only possible to detect ES-cells for instance by means of their fluorescence emission under fluorescence excitation, but also those cells that are under the control of the cell-specific and/or development-specific promoter can be selected at the same time and highly specifically. With this rather elegant method a high enrichment of specific cells that are active in a particular developmental stage or are typical for a specific tissue is possible. A particularly important example is here the enrichment of cardiomyocytes derived from ES-cells. Exemplary the following advantages are mentioned:

1. The control of the resistance gene as well as the development-specific and/or cell-specific gene under one and the same promoter ensures an efficient and fast selection of the e.g. tissue specific cells, thus e.g. of heart cells. By means of FACS-analysis, it could be shown that nearly 99% of all non-heart specific cells were eliminated. This high grade of purity for a specific cell type within a highly heterogeneous cell population of embryoid bodies is also a suitable tool not only for pharmacological tests for toxic substances, for drug screening, embryotoxicological effects, screening for factors of cell proliferation in differentiation but also opens up the possibility to prepare highly purified cell populations for therapeutic applications for replacement of a tissue and the generation of tissue samples in vitro (bioengineering), respectively.

2. Although the differentiation method preferably employed according to the invention with the “hanging drop” allows cell populations with relatively stable differentiation characteristics on plating, embryoid bodies nevertheless show clear differences at the point of time of initiation of the differentiation, i.e. of the spontaneous beating. By the expression of e.g. the fluorescence gene one obtains a reliable information about the initiation of the differentiation, for instance the cardiomyogenesis, and the addition of the selection mediums occurs adjusted in time after initiation of the transcription from the cell-specific or development-specific promoter. The combination according to the invention of a gene that encodes a detectable, e.g. fluorescent protein with a selection gene, wherein both genes are under control of one promotor, allows therefore an exact timing of the addition of selection medium depending from the differentiation stage of the cells, wherein the differentiation stage is ascertainable by the practitioner by the expression of the fluorescent protein. Under in vivo conditions the use of the reporter gene is not critical, since it could not be detected anyway. But it is of importance in the experimental testing of the method (very important for establishing of purification as well as surgical methods), but potentially not applicable for therapeutic purposes because of the potential antigenicity. Alternatively, particularly for therapeutic purposes, the use of a transgenic epitope is suitable, which is not linked to an intracellular signal cascade (for example CD8 or CD4) and under control of the cell-and tissue-specific promoter, respectively. With the help of this technique highly purified cardiomyocyte preparations might be obtained after puromycin enrichment by means of MACS sorting after enrichment with e.g. percoll gradient; further the transgenic cells might be identified in vivo and in vitro by means of anti-CD8 (anti-CD4) fluorescent conjugated cell surface antibodies. At the same time the highest possible quantitative enrichment of the desired cell types can be obtained. An addition of the selection medium at random, independently of the information about the cell differentiation, would lead to a pre-mature destruction of the precursor cells or to only a low number of terminally differentiated cells.

It is assumed that the differentiation of the ES-cells into specific cell types, particularly in the natural surrounding of the respective organs, processes particularly efficiently, since in the organ surrounding area further factors are present that promote the tissue specific differentiation of the ES-cells. Indeed, we could demonstrate in our transplantation experiments that without a tissue injury (absence of differentiation factors) no ingrowing and no differentiation of transplanted embryonic heart muscle cells can be observed. Further, after transplantation into a cryoinfarcted area a significantly increased heart muscle generation can be observed using undifferentiated embryonic stem cells (in vitro only 3 to 5%, in vivo much more effective, but with the generation of tumours). Due to the high sensitivity of ES-cells without the resistance gene for antibiotics the method according to the invention can be used to introduce the transgenic ES-cells provided by the invention into the respective organ in vivo or in vitro, in which the highly efficient differentiation for example into heart cells happens. After several weeks the selection medium is than added and all cells derived from the ES-cells are systematically killed off with the exception of those that carry the resistance gene. With this approach a more efficient generation of tissue can be expected without the associated risk of a tumour development. Crucial for the system developed here is that the antibiotic resistance gene and the reporter gene are under control of the same promoter. The reason for this is that the reporter gene indicates the point in time of the onset of the cell-specific and development-specific differentiation, respectively, for example of the heart differentiation; i.e. a major part of the early heart cells is already formed and still proliferative. At this point in time the anti-biotic resistance gene is generated and thereby all cells are killed off after addition of the anti-biotic except for the cells that express the resistance gene, e.g. also for the cardiomyocytes. In DE 19727962 different promoters were used, so that this synchronisation was not given and therefore the selection was inefficient.

Instead of a double transfection a vector containing an IRES can be constructed, in which one and the same promoter, e.g. the α-MBC promoter, drives the reporter gene and the antibiotic resistance gene and therefore a single transfection is sufficient.

An important goal of the invention is of course not only the in vitro but particularly the in vivo applicability of differentiated cells provided by the method according to the invention, particularly of heart cells. To rule out that during a transplantation for instance pluripotent stem cells or germline cells that can develop into tumour cells get into the patient, the cells of one embodiment of the invention can be made more sensible for the resistance genes by over expression, for example by using of an Oct-4 promoter. This will further reduce the likelihood that pluripotent cells survive the attack by the resistance agent.

In a further embodiment of the invention, the cells can be manipulated additionally so that specific tissues are not formed. This can occur for instance by insertion of repressor elements, e.g. a doxizyclin inducible repressor element. Thereby, a possible contamination of the desired differentiated cells with pluripotent, potentially tumourigenic cells can be excluded.

In a further embodiment one can select for cells with a high rate of division by chasing a suitable promoter, for instance the chicken β-actin-promoter and by this way further reduce the possibility of survival of pluripotent cells.

In a preferred embodiment, two kinds of vectors were used to stably transfect embryonic stem cells and to select heart cells specifically from a differentiating culture of embryonic stem cells:

1. the heart α-MHC-promoter controlled resistance gene for puromycin (α-MHC-pur);

2. the heart-α-MHC-promoter controlled gene for the enhanced green fluorescent protein (enhanced Green Fluorescent Protein=EGFP) (α-MHC-EGFP).

The novelty of the present invention consists of the combined application of a resistance gene (e.g. pur) as well as for instance a live fluorescent reporter gene (e.g. EGFP) under control of one and the same, preferably heart specific promoter (e.g. α-MHC). Such an approach shows a combination of the following advantages that facilitate genetic selection, e.g. for ES derived heart muscle cells:

i) Monitoring of the differentiation of embryonic stem cells, e.g. the heart differentiation of very early developmental stages by detection of a specific, e.g. heart specific fluorescence (Kolossov et al., 1998).

ii) Optimisation of the time for the onset of drug application by defining the fluorescence as an indicator of, e.g. α-MHC-promoter-activity, that controls the resistance gene.

iii) Visual control of the processes of the drug selection by live monitoring of the ratio between fluorescent and non fluorescent cell fractions. Feasibility of a quanitative estimation of the level of specific cell type enrichment by means of Fluorescence Activated Cell Sorting (FACS).

iv) The preferred used of the pur gene under control of a preferably heart specific promoter allows a highly effective heart specific selection by puromycin in adherent as well as in suspension cultures of differentiating ES-cells since puromycin has a faster and stronger toxic effect on non resistant cells than other known selection agents, e.g. G418 and hygromycin.

In contrast to other antibiotic resistance genes the present highly efficient and very fast selection by puromycin was surprising. Furthermore, it was a totally unknown observation for ES-cells.

v) The possibility of monitoring the fate of introduced selected cells after transplantation by simple application of e.g. EGFP-fluorescence detection. This is of fundamental importance for the establishment of novel surgical techniques.

The invention contains several aspects that having regard to the state of the art could not be expected with a reasonable expectation of success.

1. First, the simplicity with which the ES-cells could be double transfected was surprising.

Our experiments demonstrated that in most transfected clones an effective transfection with both constructs took place.

2. Furthermore, it turned out to be crucial for the efficiency of the antibiotic resistance that the selection agent is added during the early phase of the differentiation, particularly of the differentiation of heart cells. Thereby, the efficiency of the e.g. cardiomyogenesis in vitro is apparently increased, most likely because the surrounding cells release negative signals. Early phase refers to 2-4 days after plating, particularly in the hanging drop method with plating, a stage that still shows early patterns with respect to proliferation (cells are still proliferative) as well as ion channel expression (if channel is still expressed in all cardiomyocytes, all cell types including ventricular cells express this ion channel and beat spontaneously) and their regulation (basal inhibition of the L-type Ca²⁺ influx by means of muscarinergic agonists of the nitrogen monoxide system).

3. Also surprising was the highly efficient action of puromycin that led to 99% elimination of all non-cardiomyocytes within 12-24 hours.

4. The crucial advantage of the present invention is the possibility of the selection also of non plated Bs and in stirring cultures, respectively, since here the killed cells can be washed out without problems and thereby pure cell type specific cultures from ES-cells can be obtained for the first time. Partly the elimination of non vital cells is improved by enzymatic digestion (e.g. trypsin, collagen). The efficiency of this method could be further validated by cardiomyocytes in non plated EBs, which begin to contract anew when in a cell network.

In a further embodiment of the invention, the embryonic stem cells are stably transfected with two sets of vector selection systems. The first vector contains the information for a first non-cell-damaging detectable, e.g. fluorescent protein and/or for a first resistance gene, and both genes are under the control of a first cell-specific or development-specific promoter, which is operably linked with the afore mentioned genes. A second vector contains the information for a second non-cell-damaging, detectable e.g. fluorescent protein and/or for a second resistance gene and both genes are under the control of a second cell-specific or development-specific promoter, which in turn is operatively linked with these genes. Alternatively to electroperation a highly efficient transfection can be made also with viruses or as well with lipofection. Particularly worth mentioning with respect to the successful transplantation at the heart is the in vitro selection of mesodermal precursor cells. These cells are selected in accordance with above-mentioned procedure by preferably brachyuria, NIcx2.5 and ANT promoter switch elements expressing fluorescent and resistance genes and selected and transplanted afterwards. Instead of the fluorescence genes other genes of the above described detectable proteins can, of course, be used. This procedure is ideally suited to produce a larger amount of purified precursor cells, that e.g. after implantation into an injured myocardium differentiate under native differentiation factors in situ into heart cells without any hazard.

Furthermore, this approach is ideally suited to test different active agents/differentiation factors in vitro that differentiate the mesodermal precursor cells into the different specialised cell types (i.a. immunological cells, smooth- and skeletal muscle cells as well as endothelial cells). Therefore, the system is ideally suited for the testing of differentiating factors, pharmacological and otherwise active agents (i.a. toxicological substances, environmental toxins, chemicals of daily use, testing for teratogenic/embryo toxicological effects and for pharmacology).

Further, apart form the in vitro differentiation and selection a completely new procedure for tissue regeneration was established. On the one hand, the advantage is exploited that in damaged tissue (e.g. in heart infarct area) native factors are released, which positively influence the heart cell differentiation. Therefore, e.g., transgenic embryonic stem cells are generated, wherein on the one hand for instance particularly the puromycin resistance gene is under control of, e.g., the α-MHC promoter (α-MHC-puromycin) to exclude the possibility of a tumour generation. Additionally, the poxvirus driven tk-element is used. Therefore, the embryonic stem cells are triple transfected with an ubiquitary expressed promoter (e.g. chicken β-actin promoter) and the anti-tk element under control of the α-MHC promoter. Subsequently, the transgenic differentiating ES cells are injected into the damaged heart area. The intrinsic factors promote a highly efficient heart development of the ES-cells in vivo in contrast to the in vitro differentiation capacity. After 14-21 days selectively all non cardiomyocytes are selected by means of the combined systematic application of the resistance agents, e.g. puromycin and the virostatica gancyclovir. By this combined selection the potential survival of undifferentiated ES-cells and the risk of tumourigenicity is avoided. Furthermore, a considerably more efficient heart muscle development is achieved.

The invention is illustrated below by means of examples and attached figures. The figures show:

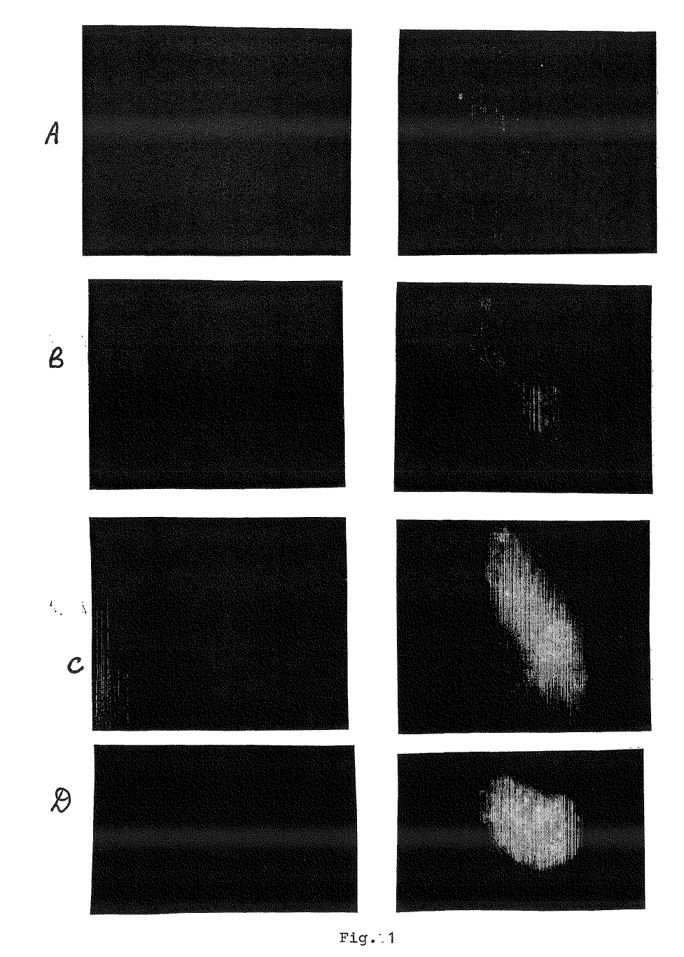

FIG. 1: Combined transmission/fluorescent light microscopic images of plated EBs that are derived from pαMHC-pur transgenic ES-cells, on the 10. (A), 11. (B), 12. (C) and 14. (D) day of development after 1, 2, 3 and 5 days, respectively, of the puromycin treatment.

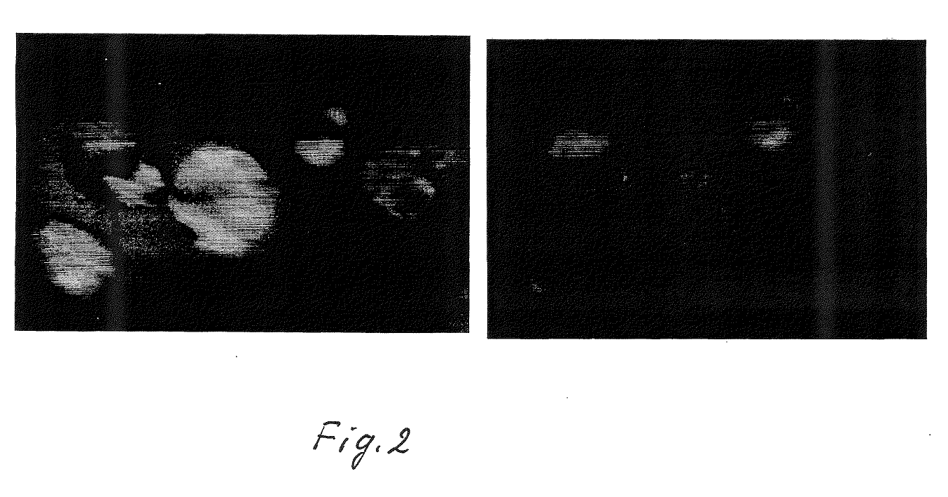

FIG. 2: Combined transmission/fluorescent light microscopic images of a suspension culture of pαMHC-pur EGFP/pαMHC-pur EBs on the 19, day of development after 10 days of puromycin treatment.

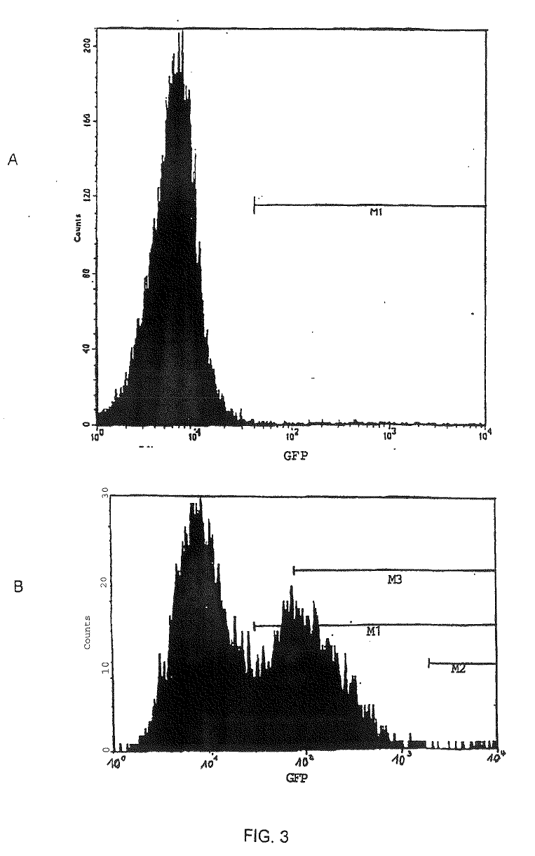

FIG. 3: (A) FACS-profile of the dissociated, 16 days old EBs that are derived from pαMHC-EGFP transgenic ES-cells. All EBs contained large beating and fluorescent heart muscle cell cluster. EGFP positive cells (M1) constitute less than 1% of the whole cell population.

(B) FACS-profile of the dissociated 22 days old EBs that are derived from cotransfected pαMHC-EGFP and pα-MHC-purES-cells after 13 days of puromycin treatment. EGFP positive cells (ml) constitute 42-45% of the whole cell population.

FIG. 4: Protocol for the preparation of embryoid bodies

### EXAMPLE 1

Materials and Methods.

Vectors.

The vector containing the regulatory 5.5 kb fragment of the Maus α-MHC-Genes was provided by Dr. J. Robbins (Children Hospital Medical Center, Cincinnati, USA) (Gulick et al., 1991).

The fragment was cut from the vector with BamHI and SalI, provided with blunt-ends and cloned into the SmaI-site of the multiple cloning site of the pEGFP-1 vector (contains the coding sequence for EGFP, the enhanced version of GFP and the Neo-cassette for the G418-resistance) (CLONTECH Laboratories, Palo Alto, Calif., USA). The correct “tail-to-head”-orientation of the promoter with respect to the coding sequence of EGFP in the resulting vector was controlled and confirmed by EcoRI-Restriktion.

The coding part of the Pur-gene (HindIII-SalI-fragment) was blunt-ligated into the pαMHC-EGFP in place of the EGFP coding sequence cut out by BamHI-AflII (ligation of blunt-ends). The correct alignment and orientation, respectively, in the resulting vector pα-MHC-Pur was confirmed by SmaI and ClaI-StuI-restrictions.

Cell Culture. Transfection and Selection Methods.

All stages of the propagation and the selection of ES-cell clones were carried out in ES-cell-propagation medium that consisted of the following: glucose rich DMEM medium supplemented with:

non-essential amino acids (0.1 mM). L-glutamine (2 mM), penicillin and streptomycin (5 μg/ml), β-mercaptoethanol (0.1 mM), LIF (ESGRO™) (500 u/ml), fetal calve serum (FCS) (15% VN).

Both vectors, pαMHC-EGFP and pαMHC-Pur, were linearised by HindI-II-Restrictase before cotransfection by electroporation of the ES-cells (D2 line). Conditions for electroporation:

cells: 4 to 5×10⁶ in 0.8 ml PBS (Ca²⁺, Mg²⁺ free)

vector-DNA: 20-40 μg;

electroporation-cuvette: 0.4 cm (Bio-Rad Laboratories, Hercules, Calif., USA);

electroporator: Gene Pulser™ (Bio-Rad Laboratories);

electrical impulse conditions: 240V, 500 μF.

After the electrical impulse, the cell suspension was cooled on ice for 20 minutes and then transferred onto a 10 cm tissue-quality petri dish together with a G418-resistant fibroblast-feeder layer in 10 ml ES-cell propagation medium. 2 days later, Geneticin G418 (GibcoBRL) was added, 300 μg/ml for the selection of G418-resistant cells. The medium with G418 (300 μg/ml) was exchanged every second day. After 8-10 days selection the drug resistant colonies appeared. The colonies were taken out, separately trypsinised in 0.1% Trypsin/EDTA solution and plated onto 48-well plates with G418 resistant fibroblast feeder layer in ES-cell propagation medium and G 418 (300 μg/ml). After 2-4 days of growth, the ES-cell clones were trypsinised subsequently and propagated in 24 well-plates and thereon on 5 cm tissue petri dishes. G418 (300 μg/ml) and G418 resistant fibroblast-feeder layer were present in all stages of the ES-cell clone propagation.

Differentiation of ES-Cells and Heart Specific Selection.

All steps of the differentiation protocol were carried out in “differentiation medium”, that consisted of all components of the previously mentioned “ES-cell propagation medium”, except for LIF, and in which the 15% FCS were substituted by 20% FCS. After the propagation, the selected G418 resistant ES-clones were trypsinised and resuspended in “differentiation medium” up to a final concentration of 0.020 to 0.025×10⁶ cells/ml. Subsequently, hanging drops were formed by arranging of 20 μl of this suspension (400 to 500 cells) on the lids of bacteria petri dishes (Greiner Labortechnik, Germany). After 2 days of incubation at 37° C. and 5% CO₂ the ES-cells formed aggregates or “embryoid bodies”, which were washed out in bacterial petri dishes with differentiation medium and were incubated for additional 5 days. After that, the embryoid bodies were plated separately onto 24-well tissue quality plates preconditioned with gelatine in differentiation medium. In parallel experiments, a number of embryoid bodies were left in suspension, where they were treated like the plated ones.

In all growth, differentiation and drug selection stages, the EBs were monitored under the fluorescence microscope using a FITC filterset (Zeiss, Jena, Germany).

In typical experiments, the application of the selective drug puromycin (1-2 μg/ml) was started on day 9-10 of the development, when the first EGFP-fluorescence was monitored. The medium with the active substance was exchanged every 2-3 days.

FACS-Analysis

For the FACS-analysis, 10 to 20 embryoid bodies from different developmental and selection stages were washed with PBS and then dissociated into a single cell suspension by trypsin treatment for 2-3 minutes (120 μl trypsin/EDTA-solution). Subsequently, 1 ml DMEM+20% FCS of the single cell suspension were added. After centrifugation (1000 upm) for 5 minutes, the cells were resuspended in 0.5 to 1.0 ml PBS that contained Ca²⁺ (1 mM) and Mg²⁺ (0.5 mM).

The GFP expression of cells of different age derived from embryonic stem cells was determined with a FACSCalibur™ flowcytometer (Becton Dickinson, BRD), that was equipped within 488 nm argon ion laser (15 mW). The cells were resuspended in PBS (pH 7.0, 0.1% BSA) up to a concentration of 5×10⁵ cells/ml and then analyzed with the FACScalibur™ with a minimum of 10.000 viable cells that were extracted for each example. The emitted fluorescence of the GFP was measured at 530 nm (FITC-bandfilter). The live gating was carried out by adding propidium iodine (2 μg/ml) to the samples immediately before measurement. Necrotic cells with a positive propidium iodine (PI) staining (885 nm bandfilter) showed a higher side-scattering-signal (SSC) in comparison to viable-PI-negative cells. Non viable cells were excluded from the subsequent assays, by letting cells with low SSC-signals pass through. Non-transfected ES-cells of the cell line D3 were used as negative controls. Assays were carried out using the CellQuest software (Becton Dickinson).

Results

ES-cells that were transgenic regarding the pαMHC-EGFP as well as the pαMHC-pur-vectors were cultivated and used in the heart differentiation protocol. All tested clones showed no microscopically verified EGFP-fluorescence in the ES-cell state and after forming EBs up to the day of plating (7 days after the formation of “hanging” drops). On the first to second day after plating (8-9 days old EBs) the first EGFP-fluorescent areas appeared, which usually started beating spontaneously one day later. Remarkably, the vast majority of EB-cells outside the beating clusters showed no microscopically measurably fluorescence level, indicating a high tissue specificity of the EGFP-expression during the ES-cell cardiomyogenesis.

After application of puromycin (typically starting on day 9-10 of the development) the first significant changes in the morphology of the plated EBs was detected within 12 hours (by means of a long term monitoring system) on the next day: The cell-growth that surrounded the beating clusters of the EGFP-fluorescent cells was reduced dramatically and the intensity of beating of the cluster that had been freed of the surrounding cell-growth did intensify unexpectedly (FIG. 1A). During the next two days, these changes progressed and showed a serious destruction of non-fluorescent cell-masses as well as a compaction of fluorescent heart-clusters with intensive contractile activities (FIG. 1B,C). Already on day 1 of the puromycin-treatment, some of the embryoid bodies had disposed of the surrounding non-fluorescent cells visually and looked like isolated, beating and fluorescent clusters (FIG. 1D). Even after 4 days of development and after 18 days of puromycin treatment, these isolated clusters showed still an intensive contractile activity, whereas in their untreated counterparts this activity typically stopped at day 17 to 20 of the development.

The increased EGFP-fluorescence as well as the sustained contractile activity was monitored in puromycin treated bodies in suspension-culture in comparison with untreated counterparts. After more than 3 weeks of development and two weeks of puromycin treatments, the suspension of embyoid bodies contained a lot of intensely fluorescent and contractile embryoid bodies, of which some presented as visibly and collectively beating fluorescent clusters (FIG. 2). These results clearly show that cardiomyocytes can be kept alive without the surrounding cells and differentiate. The spontaneous beating further shows the functional integrity of the selected heart-muscle cells. The crucial advantage however, was the rapidness of the puromycin selection, that led to a 99% destruction of all non-cardiomyocytes during 12-24 hours after application.

The FACS-analysis demonstrated a high effectiveness of the puromycin selection of the transgenic ES-cells used. While the EGFP-fluorescent cells represent only about 1% of the whole cell population of untreated cells that contained a pαMHC-EGFP-vector, the puromycin treatment of differentiating embryonic stem cells, that were transgenic with regard to pαMHC-EGFP as well as pαMHC-pur vectors led to a 42-45% ic enrichment of the cell population by EGFP-fluorescent cells (FIG. 3). The simple calculation shows that already 97-99% of the whole non-cardiogenic cell population was effectively killed during the puromycin treatment of the suspension culture of transgenic ES cells. The still existing fraction of puromycin-resistant non- or weekly fluorescent cells (FIG. 3) could be explained by non-specific activity of the pαMHC-promotor in some of the non-cardiogenic cells. Such a fraction was eliminated by either higher concentration of puromycin or by FACS-sorting methods.

### EXAMPLE 2

As initial vector pIRES2-EGFP (Clontech Laboratories, Palo Alto, Calif.) was used. This vector contains an internal ribosome-entry site (IRES) of the encephalomyocarditis virus between the multiple cloning-site (MCS) and the EGFP-gene. This allows that the puromycin resistance as well as the EGFP-gene are translated separately from one single bicistronic mRNA. The pIRES2-EGFP vector was blunt ended with the restriction enzymes AseI and ECO47III and religated in order to delete the cytomegalovirus immediate early (CMV-IV) promoter. The resulting vector was digested with SmaI and ligated with the α-MCH-pur-cassette, which had been cut out of the above described α-MHC-pur vector by SacI and ClaI. The correct orientation of the obtained pα-MHC-IRES-EGFP (pα-PIG) vector was verified by digest with SacI/SmaI.

ES-cells (D3-cell line) were transfected with pα-PIG; the following G418-selection, the propagation and differentiation of the obtained stable clones was carried out as already described in Example 1.

After carrying out the standard differentiation protocol, one could demonstrate beating clusters of EGFP positive heart cells between the 8^{th} and the 9^{th} day of development, where upon puromycin 5 μg/ml was added. After the first three to four days of the puromycin treatment, the embryoid bodies (EBs) contained mainly EGFP-positive, intensively beating clusters of heart cells; non-heart cells detached and were eliminated when the medium was changed. The same result could be achieved by letting the EBs grow entirely in suspension culture and carrying out the resistance treatment with the antibiotic. A FACS-analysis showed an enrichment of at least 70% (flowcytometry using EGFP as read out) in the so obtained cell culture. The arrangement of reporter gene and resistance gene on one vector under control of one promoter, preferably in combination with an IRES, is therefore excellently suited for the production of differentiated embyonal stem cells that are as far as possible free of undifferentiated stem cells. The same applies of course to germline cells and adult stem cells, respectively, and not only to embryonic stem cells. In particular, it could be shown by this example that an outstandingly high tissue specificity is achievable for heart cells developing from ES-cells.

Validity of the Puromycin Selection Protocol

The puromycin selection method was subsequently tested in an autologous mouse model, wherein an injury of the heart was simulated, and could thereby be validated. For this purpose, a mouse transplantation model was used, in which embryonic stem cells or heart cells obtained by in vitro differentiation of ES-cells (10.000-100.000 cells) were injected into a recipient, whose heart was partially damaged by low temperature treatment. The development of tumours was morphologically examined by means of the whole mouse, of the isolated heart and of tissue slides; these examinations were carried out at different points of time after the operation over a period of two days up to two months. This approach allows an exact evaluation of the tumour potential of the different cell preparations. On injection of non-differentiated ES-cells into the cryo-injury (100.000 cells) large tumours developed in the mice. 10 days after the operation the animals died of these tumours. But tumours developed also, when ES-cells were differentiated in vitro into heart cells and the beating areas, which are typical for cardiomycytes derived from ES-cells, were separated, isolated and 10.000 to 50.000 cells thereof were injected into the mice. This demonstrates the high tumour potential of embryonic stem cells in the heart and the high demands that have to be made on a highly specific selection method.

In the next experiment, transgenic ES-cells, that were stably transfected with a construct of the invention (reporter gene and resistance gene under the control of one promoter on one vector) were put through a puromycin treatment for five to seven days after demonstration of EGFP expression. In a large test series of more than 25 surgically treated mice that had all been put through the cryo-treatment on the heart described above, no development of tumours could be observed even after several month, if these puromycin-resistant ES-cell derived cells (10.000 to 50.000 cells) were injected into the injured mice heart area (double transfections constructs). Indeed, we succeeded in identifying the cells after the transplantation and it could be demonstrated clearly that the cells could be transplanted successfully and that they differentiated to terminal differentiated cardiomyocytes. These experiments show clearly the capability of the technique described in accordance with the invention to enrich in vitro-differentiated. cells efficiently and to obtain a population that does not contain any undifferentiated ES-cells. Having regard to the high tumourigenicity shown here of ES-cell derived cells in the heart, this efficiency is particularly remarkable.

### CONCLUSIONS

1. Stable transgenic embryonic stem cell clones, that were cotransfected with the expression-vector pαMHC-EGFP and pαMHC-pur were prepared.

2. A puromycin treatment of the transgenic embryonic stem cells during the differentiation in vitro showed a high efficiency of cardiospecific selection in comparison to a hygromycin treatment of previously generated pαMHC-Hyg ES-cell lines (data not shown).

3. The selected differentiated cells showed a higher degree of morphological and functional viability and longevity as their untreated counterparts, which suggests that the genetic selection approach efficiently liberates differentiating embryonic stem cells from negative influences of the surrounding cells.

4. The combined use of live-fluorescence reporter and drug resistance genes under a common cell type-specific promoter allowed the tight monitoring and quantification of the whole procedure, including the differentiation and the cell type-specific selection. The resulting cells are applicable to further transplantation experiments, which allows the monitoring of the introduced cells.

4. The approach presented can be applied to any cell type specific selection in an ES-cell differentiation system, if a highly specific promoter for the respective cell type or a specific stage of development is identified and cloned. In principal, the system allows the combined use of two different promoters with respective two colored in vitro fluorescent proteins, for example the yellow (EYEP) and cyan (blue) (ECFP) versions of EGFP, and two drug resistance genes. Such an approach might increase the selectivity and efficiency of the whole procedure. The embryonic stem cells provided by the invention, preferably embryoid bodies, can be used for toxicological tests of substances, for example heavy metals and pharmaceuticals, (see also the listing above). For this purpose, embryonic stem cell cultures are utilised using the double vector constructs and the selection agents is added after the start of the cell typical differentiation (detection of the fluorescence). After the cell purification or already during the ES-cell cultivation, the different substances to be tested are added to the cell culture and at different points in time the fluorescent single cells and the overall fluorescence, respectively, is measured by different readout methods (e.g. flowcytometry, fluorescencereader) in comparison to the controls.

The embryonic stem cells provided by the invention can be used for the generation of transgenic non-human mammals with cell specific or development specific expression of the fluorescent protein. Here the described ES-cells of the invention are introduced into blastocysts of non human mammals. In the next step the blastocysts are transferred into foster mothers as chimeras, that become homozygous by backcrossing, and thereby transgenic non-human mammals are generated.

In a further embodiment of the invention the transgenic embryonic stem cells are used in form of a pharmaceutical composition for transplantation purposes. For this purpose, highly purified embryonic stem cell derived cultures are needed, since it is known that a contamination with undifferentiated proliferating stem cells leads to tumour generation. Accordingly, the method described herein is ideally suited to obtain highly purified ES-cell derived cell specific cultures that are ideal for transplantation (Klug et al., 1996).

Finally, it should be stressed again, that the invention illustrated above by means of embryonic stem cells is also applicable to embryonic germline cells and to adult stem cells.

The present invention discloses a system for the cell- and development-specific selection of differentiating embryonic and adult stem cells or embryonic germline cells by the combined use of resistance and detectable reporter genes under common control of a cell- and/or development-specific promoter.

### REFERENCES

Anderson, D. J., Gage, F. H., and Weissmann, I. L. (2001). Can stem cells cross lineage boundaries? Nat. Med. 7, 393-395.

Doetschman, T. C., Eistetter, H., Katz, M., Schmidt, W., and Keinler, R. (1985). The in vitro development of blastocyst-derived embryonic stem cell lines: formation of visceral yolk sac, blood islands and myocardium. J. Embryol. Exp. Morphol. 87, 27-45.

Gage, F. H. (2000). Mammalian neural stem cells. Science 287, 1433-1438.

Heim, R. and Tsien, R. Y. (1996). Engineering green fluorescent protein for improved brightness, longer wavelengths and fluorescence resonance energy transfer. Curr. Biol. 6, 178-182.

Kallmeier, R. C., Somasundaram, C., and Babij, P. (1995). A novel smooth muscle-specific enhancer regulates transcription of the smooth muscle myosin heavy chain gene in vascular smooth muscle cells. J. Biol. Chem. 270, 30949-30957.

Klug, M. G., Soonpaa, M. H., Koh, G. Y., and Field, L. J. (1996). Genetically selected cardiomyocytes from differentiating embryonic stem cells form stable intracardiac grafts. J Clin. Invest. 98, 216-224.

Kolossov, E., Fleischmann, B. K., Liu, Q., Bloch, W., Viatchenko-Karpinski, S., Manzke, O., Ji, G. J., Bohlen, H., Addicks, K., and Hescheler, J. (1998). Functional characteristics of ES cell-derived cardiac precursor cells identified by tissue-specific expression of the green fluorescent protein. J Cell Biol 143, 2045-2056.

Lints, T. J., Parsons, L. M., Hartley, L., Lyons, I., and Harvey, R. P. (1993). Nkx-2.5: a novel murine homeobox gene expressed in early heart progenitor cells and their myogenic descendants [published erratum appears in Development 1993 November; 119(3):969). Development 119, 419-431.

Lothian, C. and Lendahl, U. (1997). An evolutionarily conserved region in the second intron of the human nestin gene directs gene expression to CNS progenitor cells and to early neural crest cells. Eur J Neurosci. 9, 452-462.

Muldoon, R. R., Levy, J. P., Kain, S. R., Kitts, P. A., and Link, C. J., Jr. (1997). Tracking and quantitation of retroviral-mediated transfer using a completely humanized, redshifted green fluorescent protein gene. Biotechniques 22, 162-167.

Muller M, Fleischmann B. K., Seibert S, Ji G. J., Endl E, Middeler G, Mueller O J, Schlenke P, Frese S. Wobus A M, Hescheler J, Katus, H. A., and Franz, W. M. Selection of ventricular-like cardiomyocytes from ES cells in vitro. FASEB J. 2000. Ref Type: In Press

O'Brien, T. X., Lee, K. J., and Chien, K. R. (1993). Positional specification of ventricular myosin light chain 2 expression in the primitive murine heart tube. Proc. Natl. Acad. Sci. U.S.A. 90, 5157-5161.

Prockop, D. J. (1997). Marrow stromal cells as stem cells for nonhematopoietic tissues. Science 276, 71-74.

Sartorelli, V., Webster, K. A., and Kedes, L. (1990). Musclespecific expression of the cardiac alpha-actin gene requires My9D1, CArG-box binding factor, and Spl. Genes Dev. 4, 1811-1822.

Shamblott M J, Axelmann J, Wang S, Bugg E M, Littlefield J W, Donovan R T, Blumenthal P D, Huggins G R, and Gearhart J D (1998). Derivation of pluripotent stem cells from cultured human primordial germ cells. Proc. Natl, Acad. Sci. U.S.A. 95, 13726-13731.

Thomson J A, Itskovitz-Eldor J, Shapiro S S, Waknitz M A, Swiergiel J J, Marshall V S, and Jones J M (1998). Embryonic stem cell lines derived from human blastocysts. Science 282, 11451147.

Wartenberg, M., Donmez, F., Ling, F. C., Acker, H., Hescheler, J.,and Sauer, H. (2001). Tumor-induced angiogenesis studied in confrontation cultures of multicellular tumor spheroids and embryoid bodies grown from pluripotent embryonic stem cells. FASEB J 2001. April; 15. (6.):995. -1005. 15, 995-1005.

Wobus, A. M., Wallukat, G., and Hescheler, J. (1991). Pluripotent mouse embryonic stem cells are able to differentiate into cardiomyocytes expressing chronotropic responses to adrenergic and cholinergic agents and Ca2+ channel blockers. Differentiation. 48, 173-182.

1-31. (canceled)

32. A method for the toxicological testing of substances on differentiating or differentiated cardiomyocytes comprising:
(i) providing a cell culture of differentiating cardiomyocytes or differentiated cardiomyocytes, wherein stem cells are differentiated into cardiomyocytes by:
(a) introducing into said stem cells a vector comprising DNA sequences encoding a reporter gene and a puromycin-resistance gene both operably linked to a single heart-specific promoter, wherein an IRES sequence is located between the reporter gene and the puromycin gene, and wherein said reporter gene encodes a non-cell damaging detectable protein or epitope thereof;
(b) culturing said cells in the form of embryoid bodies under conditions allowing differentiation into cardiomyocytes;
(c) detecting live cells expressing said reporter gene;
(d) upon first detection of said reporter gene adding puromycin at a concentration of greater than or equal to 1 μg/ml for the selection of cells expressing said puromycin-resistance gene; and
(e) recovering differentiating cardiomyocytes or differentiated cardiomyocytes, wherein 99% of all non-cardiomyocytes are eliminated;
(ii) introduction of substances, whose toxic or non-toxic properties are to be tested, into the cell culture; and
(iii) quantitatively and/or qualitatively determining the fluorescence of the cells obtained in comparison with cells that were cultivated without the substance to be tested.

33. The method of claim 32, wherein said vector is introduced by a method selected from the group consisting of transfection and viral vectors.

34. The method of claim 32, wherein said vector further comprises DNA sequences encoding a second resistance gene under control of a constitutively active promoter.

35. The method of claim 32, wherein the selected cells sorted for additional enrichment.

36. The method of claim 32, wherein said detectable protein or epitope thereof is selected from the group consisting of green fluorescent protein (GFP), enhanced green fluorescent protein (EGFP), red fluorescent protein (RFP), blue fluorescent protein (BFP), yellow fluorescent protein (YFP) and cyan fluorescent protein (CFP).

37. The method of claim 33, wherein said transfection is by a method selected from the group consisting of electroporation and lipofection.

38. The method of claim 32, wherein puromycin is added 8 to 10 days after development.

39. The method of claim 32, wherein the substances are added to the cell culture during step (b).

40. The method of claim 32, wherein the vector containing cells are selected by a method comprising: adding a second selection agent for the selection of stably transfected cells expressing said second resistance gene prior to said detecting of cells expressing said reporter gene
